(19) United States
(12) Patent Application Publication
(10) Pub. No.: US 2025/0188517 A1
(43) Pub. Date: Jun. 12, 2025

(54) COMPOSITIONS AND METHODS RELATED TO NUCLEIC ACID SENSORS

(71) Applicant: Arizona Board of Regents on behalf of Arizona State University, Scottsdale, AZ (US)

(72) Inventors: Rizal F. Hariadi, Scottsdale, AZ (US); Gde Bimananda Mahardika Wisna, Scottsdale, AZ (US); Ranjan Sasmal, Scottsdale, AZ (US)

(21) Appl. No.: 18/844,872

(22) PCT Filed: Mar. 7, 2023
(86) PCT No.: PCT/US23/63860

§ 371 Date: Sep. 6, 2024

Related U.S. Application Data
(60) Provisional application No. 63/317,424, filed on Mar. 7, 2022.

(51) Int. Cl. C12Q 1/6825

(52) U.S. Cl. CPC C12Q 1/6825

(57) ABSTRACT

The present disclosure provides compositions and methods related to nucleic acid sensors. In particular, the present disclosure provides membrane-spanning nucleic acid sensors, signal transducers, and molecular amplifiers for lysis-free detection of internal nucleic acids.

### CROSS REFERENCE TO RELATED APPLICATIONS

This application claims priority to and the benefit of U.S. Provisional Patent Application No. 63/317,424 filed Mar. 7, 2022, which is incorporated herein by reference in its entirety for all purposes.

### STATEMENT REGARDING FEDERAL FUNDING

This invention was made with government support under grant number AI144247 awarded by the National Institutes of Health. The government has certain rights in the invention.

### SEQUENCE LISTING

The text of the computer readable sequence listing filed herewith, titled “SKYSG-40581-601”, created Mar. 7, 2023, having a file size of 12,686 bytes, is hereby incorporated by reference in its entirety.

### FIELD

The present disclosure provides compositions and methods related to nucleic acid sensors. In particular, the present disclosure provides membrane-spanning nucleic acid sensors, signal transducers, and molecular amplifiers for lysis-free detection of internal nucleic acids.

### BACKGROUND

The detection of nucleic acid targets non-invasively inside vesicles enclosed by lipid bilayer membranes, such as exosomes and cells, is of great interest, as it can lead to many applications in both medicine and many branches of biomedical science. Many of these applications can take advantage of the various features of nucleic acids. For example, the stem-loop structure or the hairpin structure of DNA or RNA occur naturally, and this structure is very important, since it forms the building blocks for RNA secondary structures that function as recognition sites for proteins and as nucleation sites for RNA folding. The different lengths and sequences of loops and stems, in addition to the salt concentration in solution, affect the structural stability of the hairpin, in addition to the thermodynamics and the kinetics of conformational change in folded and unfolded states. Furthermore, the simple yet powerful structure has been applied to, for example, molecular beacons, molecular computations, and hairpin chain reaction (HCR) amplification, among others uses.

Additionally, toehold mediated strand displacement (TMSD) refers to a process in which a DNA strand in a DNA helix structure called the protector strand can be displaced and replaced by an invader strand that is complementary to the other strand in the original helix structure. The other strand in the original helix structure is called the original strand, which has an overhang called a “toehold” that assists the invading strand in dislodging and replacing the protector strand. The TMSD process has many applications for DNA molecular machines, DNA computing, DNA sensing, and programmable DNA nanostructures, among others. Moreover, hairpin chain reaction (HCR) is a powerful enzyme-less isothermal amplification method based on two (or more) metastable monomer hairpins. To trigger the polymerization of the monomers, an initiator strand is introduced. HCR programmability was exploited for many applications for DNA and RNA detections, in addition to RNA imaging in fixed cells.

### SUMMARY

Embodiments of the present disclosure include a nucleic acid sensor comprising a double-stranded stem domain comprising at least one hydrophobic tag, at least one toehold domain positioned at an end of the double-stranded stem domain, and optionally, a hairpin domain positioned at an end of the double-stranded stem domain opposite of the toehold domain.

In some embodiments, the sensor comprises two toehold domains positioned at both ends of the double-stranded stem domain.

In some embodiments, the sensor does not comprise a hairpin domain.

In some embodiments, the sensor comprises two separate nucleic acid molecules having complementary sequences that form the double-stranded stem domain, and wherein each of the separate nucleic acid molecules comprises a toehold domain.

In some embodiments, the sensor comprises one toehold domain and one hairpin domain at opposite ends of the double-stranded stem domain.

In some embodiments, the sensor comprises a single nucleic acid molecule, and wherein the single nucleic acid molecule comprises an internally complementary sequence that forms the double-stranded stem domain.

In some embodiments, the nucleic acid sensor is a DNA molecule. In some embodiments, the nucleic acid sensor is an LNA molecule. In some embodiments, the nucleic acid sensor is an RNA molecule.

In some embodiments, the at least one toehold domain is complementary to a target nucleic acid sequence.

In some embodiments, the target nucleic acid sequence is a DNA molecule or an RNA molecule.

In some embodiments, the at least one toehold domain is from about 5 to about 20 nucleotides.

In some embodiments, the stem domain is from about 10 to about 30 nucleotides.

In some embodiments, the hairpin domain is from about 5 to about 20 nucleotides.

In some embodiments, the sensor comprises at least two hydrophobic tags. In some embodiments, the at least two hydrophobic tags are positioned from about 120° to about 180° from each other. In some embodiments, the at least two hydrophobic tags are positioned from about 4 to about 6 nucleotides from each other.

In some embodiments, the sensor comprises at least three hydrophobic tags. In some embodiments, the at least three hydrophobic tags are positioned from about 90° to about 120° from each other. In some embodiments, the at least three hydrophobic tags are positioned from about 2 to about 4 nucleotides from each other.

In some embodiments, the at least one hydrophobic tag is positioned such that the at least one hydrophobic tag spans from about 1.5 nm to about 3.0 nm.

In some embodiments, the sensor comprises a nucleic acid sequence that is at least 90% identical to SEQ ID NOs: 1 or 2. In some embodiments, the sensor comprises a nucleic acid sequence that is at least 90% identical to SEQ ID NOs: 5, 8, or 11.

Embodiments of the present disclosure also include a composition comprising any of the nucleic acid sensors described herein.

In some embodiments, the composition comprises at least one reporter nucleic acid. In some embodiments, the at least one reporter nucleic acid comprises a sequence that is complementary to at least a portion of the optional hairpin domain. In some embodiments, the at least one reporter nucleic acid comprises a sequence capable of initiating at least one of: (i) toehold mediated strand displacement (TMSD); (ii) loop-mediated isothermal amplification (LAMP); and/or (iii) hairpin chain reaction (HCR).

Embodiments of the present disclosure also include a method of detecting a target nucleic acid using any of the sensors of described herein.

In some embodiments, the target nucleic acid is located within a membranous vesicle or cell. In some embodiments, the method comprises detecting the target nucleic acid without lysis of the membranous vesicle or cell.

In some embodiments, the target nucleic acid is DNA or RNA.

In some embodiments, detecting the target nucleic acid comprises an amplification step. In some embodiments, the amplification step comprises at least one of toehold mediated strand displacement (TMSD), loop-mediated isothermal amplification (LAMP), and/or hairpin chain reaction (HCR).

In some embodiments, the method comprises a target detection step.

In some embodiments, the method comprises sequencing the target nucleic acid.

Embodiments of the present disclosure also include a kit comprising any of the sensors described herein, and instructions for detecting a target nucleic acid.

In some embodiments, the kit further comprises a calibrator or control. In some embodiments, the calibrator or control comprises a detection moiety. In some embodiments, the calibrator or control comprises a nucleic acid sequence that is at least 90% identical to SEQ ID NOs: 3, 4, 6, 7, 9, 10, 12, or 13.

### BRIEF DESCRIPTION OF THE DRAWINGS

FIGS. 1A-1B: Representative schematic diagrams of DNA sensors, according to one embodiment of the present disclosure.

FIGS. 2A-2B: Representative schematic diagrams of methods of using double-stranded DNA sensors for nucleic acid detection, according to one embodiment of the present disclosure.

FIG. 3: Representative confocal images showing the insertion of double stranded DNA sensors through a membrane showing green rings on the GUVs on the right panel and the detection of target strands forming red rings on the GUVs on the middle panel.

FIGS. 4A-4B: Representative schematic diagrams of methods of using hairpin DNA sensors for nucleic acid detection, according to one embodiment of the present disclosure.

FIG. 5: Representative experimental results of negative control experiments for a DNA sensor of the present disclosure.

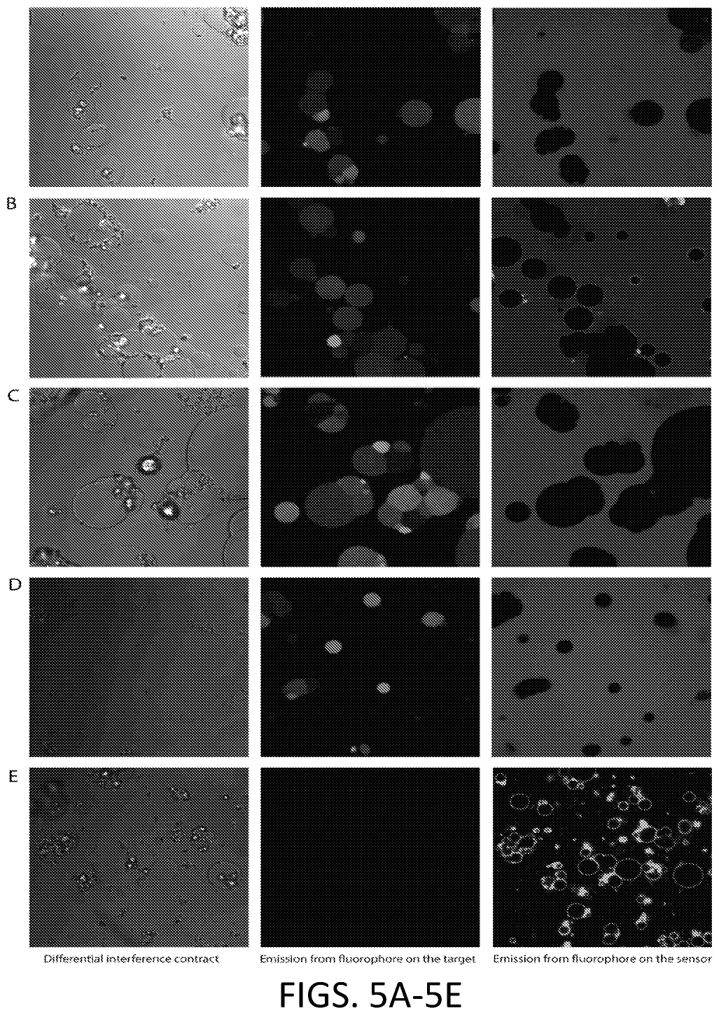

FIG. 6: Representative confocal images showing detection of internal nucleic acid targets in a vesicle with several composition ratios of POPC and Cholesterol.

FIG. 7: Representative experimental results demonstrating that double stranded sensors have minimal effects on unrelated molecules present in GUVs, causing only on average around 8.4% leakage in all three experiments.

FIGS. 8A-8B: Representative confocal images showing the insertion of hairpin DNA sensors through a membrane showing green rings on the GUVs on the left panel due to target binding to the sensor and the negative control on the right panel with scramble DNA inside the GUVs that does not produce green ring on GUVs.

FIGS. 9A-9B: TMSD schematic of hairpin DNA sensors after intravesicular target binding and opening of the sensor to enable the binding of the reporter strand on top panel and representative confocal images showing the detection of target forming green rings on GUVs on bottom left panel and binding of reporter strands on the open stem part of the sensor forming red rings on GUVs on bottom right panel.

FIG. 10: TMSD schematic of double stranded DNA sensors after intravesicular target binding allowing some part of the target to bind to the reporter added outside GUVs on top panel and representative confocal images of target detection showing red ring formation due to target binding to hairpin sensor on bottom left panel and the binding of reporter strand to the part of the target protruding outwardly from GUVs membrane due to TMSD forming blue ring on the right panel.

FIG. 11: Representative experimental result of TMSD negative control experiments for double stranded DNA sensor that shows no reporter binding indicating no TMSD across lipid membrane in the case of scramble intravesicular DNA target of the present disclosure.

### DETAILED DESCRIPTION

Embodiments of the present disclosure provide nanometer-sized biosensors fabricated from DNA that can span lipid bilayer membranes, detect internal nucleic acid targets present in vesicles, and amplify the signal from an event of detection to enable the detection of low concentration targets. This inventive subject matter of the present disclosure is based on several phenomena and technologies closely related to DNA nanotechnology and molecular biology, including DNA hairpin structure, toehold-mediated strand displacement, DNA hybridization design and free energy calculation, molecular dynamics simulation, hairpin chain reaction, and bioconjugation. Embodiments of the present disclosure utilize the multidisciplinary developments mentioned above to create a single and a double stranded DNA nanostructure with sequences, structures, and hydrophobic decoration designed in such a way that it can anchor, insert over lipid bilayer membranes, and detect internal nucleic acids of interest while allowing information to be transduced across the membrane by means of TMSD and to be amplified by means of isothermal amplification such as HCR. The use of TMSD and HCR or other isothermal amplification methods will have widespread applications for the DNA sensors of the present disclosure and their related components.

Several types of nucleic acid sensors are described herein, and each has been designed and tested to accommodate efficient insertion and anchorage across the lipid bilayer membrane of vesicles for lysis-free internal nucleic acid target detection. Transduction of signal from detection events across membranes and amplification of the information inside vesicles is carried out using toehold mediated strand displacement (TMSD), isothermal amplification (e.g., hairpin chain reaction (HCR)), and other possible amplification methods. The simple approach of single-stranded DNA and double-stranded DNA will prevent the stoichiometric issues that are often encountered in DNA biosensor synthesis. Simple applications of TMSD in conjunction with isothermal amplification provide a simple means to transfer and amplify information across the membrane using DNA biosensors for detection of low concentrations of internal nucleic acids. The detection of biomarkers present in exosomes and cells can be carried out. Additionally, DNA nanosensors non-invasive detection method can also be used to diagnose diseases and genotype cells, and targeted therapeutics.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

The term “single-stranded” oligonucleotides generally refers to those oligonucleotides that contain a single covalently linked series of nucleotide residues.

The terms “oligomers” or “oligonucleotides” include RNA or DNA sequences of more than one nucleotide in either single chain or duplex form and specifically includes short sequences such as dimers and trimers, in either single chain or duplex form, which can be intermediates in the production of the specifically binding oligonucleotides. “Modified” forms used in candidate pools contain at least one non-native residue. “Oligonucleotide” or “oligomer” is generic to polydeoxyribonucleotides (containing 2′-deoxy-D-ribose or modified forms thereof), such as DNA, to polyribonucleotides (containing D-ribose or modified forms thereof), such as RNA, and to any other type of polynucleotide which is an N-glycoside or C-glycoside of a purine or pyrimidine base, or modified purine or pyrimidine base or abasic nucleotides. Oligonucleotide” or “oligomer” can also be used to describe artificially synthesized polymers that are similar to RNA and DNA or DNA and RNA molecules with modified backbone and nucleosides, including, but not limited to, oligos of peptide nucleic acids (PNA) and locked nucleic acids (LNA).

The term “RNA analog” or “RNA derivative” or “modified RNA” generally refer to a polymeric molecule, which in addition to containing ribonucleosides as its units, also contains at least one of the following: 2′-deoxy, 2′-halo (including 2′-fluoro), 2′-amino (preferably not substituted or mono- or disubstituted), 2′-mono-, di- or tri-halomethyl, 2′-O-alkyl, 2′-O-halo-substituted alkyl, 2′-alkyl, azido, phosphorothioate, sulfhydryl, methylphosphonate, fluorescein, rhodamine, pyrene, biotin, xanthine, hypoxanthine, 2,6-diamino purine, 2-hydroxy-6-mercaptopurine and pyrimidine bases substituted at the 6-position with sulfur or 5 position with halo or C₁₋₅ alkyl groups, a basic linkers, 3′-deoxy-adenosine as well as other available “chain terminator” or “non-extendible” analogs (at the 3′-end of the RNA), or labels such as ³²P, ³³P and the like. All of the foregoing can be incorporated into an RNA using the standard synthesis techniques disclosed herein.

The terms “binding activity” and “binding affinity” generally refer to the tendency of a ligand molecule to bind or not to bind to a target. The energetics of these interactions are significant in “binding activity” and “binding affinity” because they can include definitions of the concentrations of interacting partners, the rates at which these partners are capable of associating, and the relative concentrations of bound and free molecules in a solution.

“Complementary” refers to the characteristic of two or more structural elements (e.g., peptide, polypeptide, nucleic acid, small molecule, etc.) of being able to hybridize, dimerize, or otherwise form a complex with each other. For example, a “complementary peptide and polypeptide” are capable of coming together to form a complex. Complementary elements may require assistance to form a complex (e.g., from interaction elements), for example, to place the elements in the proper conformation for complementarity, to co-localize complementary elements, to lower interaction energy for complementation, etc.

As used herein, the terms “nucleotide sequence identity” or “nucleic acid sequence identity” refers to the presence of identical nucleotides at corresponding positions of two polynucleotides. Polynucleotides have “identical” sequences if the sequence of nucleotides in the two polynucleotides is the same when aligned for maximum correspondence (e.g., in a comparison window). Sequence comparison between two or more polynucleotides is generally performed by comparing portions of the two sequences over a comparison window to identify and compare local regions of sequence similarity. The comparison window is generally from about 20 to 200 contiguous nucleotides. The “percentage of sequence identity” for polynucleotides, such as about 5, 10, 15, 20, 25, 30, 35, 40, 45, 50, 55, 60, 65, 70, 75, 80, 85, 90, 95, 98, 99 or 100 percent sequence identity, can be determined by comparing two optimally aligned sequences over a comparison window, wherein the portion of the polynucleotide sequence in the comparison window can include additions or deletions (i.e., gaps) as compared to the reference sequence for optimal alignment of the two sequences. In some embodiments, the percentage is calculated by: (a) determining the number of positions at which the identical nucleic acid base occurs in both sequences; (b) dividing the number of matched positions by the total number of positions in the window of comparison; and (c) multiplying the result by 100. Optimal alignment of sequences for comparison can also be conducted by computerized implementations of known algorithms, or by visual inspection. Readily available sequence comparison and multiple sequence alignment algorithms are, respectively, the Basic Local Alignment Search Tool (BLAST) and ClustalW/ClustalW2/Clustal Omega programs available on the Internet (e.g., the website of the EMBL-EBI). Other suitable programs include, but are not limited to, GAP, BestFit, Plot Similarity, and FASTA, which are part of the Accelrys GCG Package available from Accelrys, Inc. of San Diego, Calif., United States of America. See also Smith & Waterman, 1981; Needleman & Wunsch, 1970; Pearson & Lipman, 1988; Ausubel et al., 1988; and Sambrook & Russell, 2001.

### 2. Nucleic Acid Sensors

Embodiments of the present disclosure include compositions and methods related to nucleic acid sensors. In particular, the present disclosure provides membrane-spanning nucleic acid sensors, signal transducers, and molecular amplifiers for lysis-free detection of internal nucleic acids.

In accordance with these embodiments, the present disclosure provides a nucleic acid sensor comprising a double-stranded stem domain comprising at least one hydrophobic tag, at least one toehold domain positioned at an end of the double-stranded stem domain, and optionally, a hairpin domain positioned at an end of the double-stranded stem domain opposite of the toehold domain.

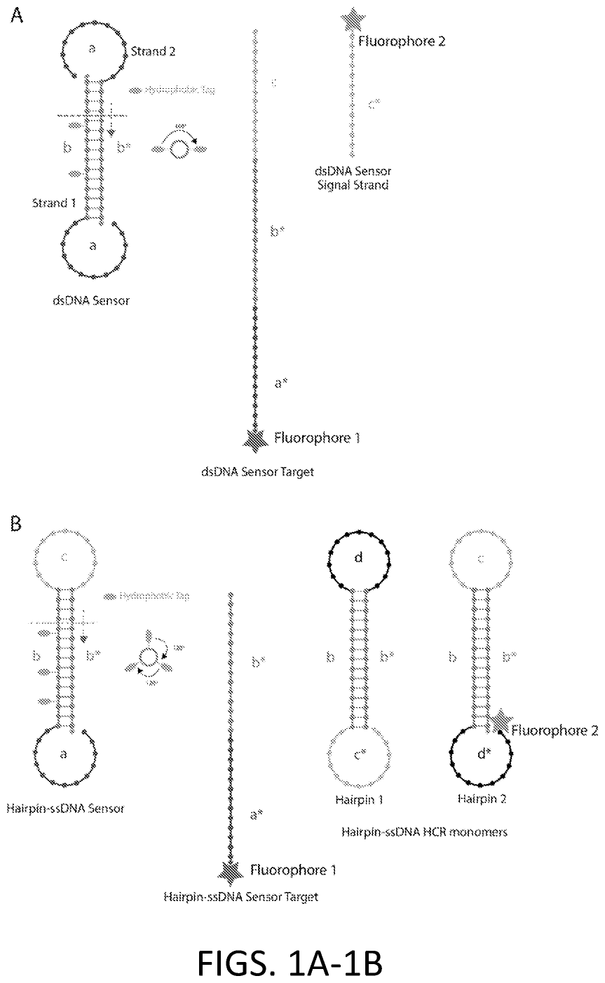

In some embodiments, the sensor comprises two toehold domains positioned at both ends of the double-stranded stem domain. In some embodiments, the sensor does not comprise a hairpin domain (e.g., FIG. 1A). In some embodiments, the sensor comprises two separate nucleic acid molecules having complementary sequences that form the double-stranded stem domain. In some embodiments, each of the separate nucleic acid molecules comprises a toehold domain (e.g., FIG. 1A). In accordance with these embodiments, the toehold domain can be open at one end (i.e., the 5′ or 3′ end of the nucleic acid molecule is unconjugated). In some embodiments, the 5′ or 3′ end of the toehold domain is conjugated to a detection moiety.

In some embodiments, the sensor comprises one toehold domain and one hairpin domain at opposite ends of the double-stranded stem domain. In some embodiments, the sensor comprises a single nucleic acid molecule (e.g., FIG. 1B). In some embodiments, the single nucleic acid molecule comprises an internally complementary sequence that forms the double-stranded stem domain (e.g., FIG. 1B). In accordance with these embodiments, the hairpin domain is closed (i.e., it comprises a continuous sequence of nucleic acids).

In some embodiments, the nucleic acid sensor is a DNA molecule. In some embodiments, the nucleic acid sensor is an RNA molecule. In some embodiments, the at least one toehold domain is complementary to a target nucleic acid sequence. In some embodiments, the target nucleic acid sequence is a DNA molecule or an RNA molecule.

In some embodiments, the at least one toehold domain is from about 5 to about 20 nucleotides. In some embodiments, the at least one toehold domain is from about 10 to about 20 nucleotides. In some embodiments, the at least one toehold domain is from about 15 to about 20 nucleotides. In some embodiments, the at least one toehold domain is from about 5 to about 15 nucleotides. In some embodiments, the at least one toehold domain is from about 5 to about 10 nucleotides. In some embodiments, the at least one toehold domain includes 5, 6, 7, 8, 9, 10, 11, 12, 13, 14, 15, 16, 17, 18, 19, or 20 nucleotides.

In some embodiments, the stem domain is from about 5 to about 30 nucleotides. In some embodiments, the stem domain is from about 10 to about 30 nucleotides. In some embodiments, the stem domain is from about 15 to about 30 nucleotides. In some embodiments, the stem domain is from about 20 to about 30 nucleotides. In some embodiments, the stem domain is from about 25 to about 30 nucleotides. In some embodiments, the stem domain is from about 5 to about 25 nucleotides. In some embodiments, the stem domain is from about 5 to about 20 nucleotides. In some embodiments, the stem domain is from about 5 to about 15 nucleotides. In some embodiments, the stem domain includes from 5, 6, 7, 8, 9, 10, 11, 12, 13, 14, 15, 16, 17, 18, 19, 20, 21, 22, 23, 24, 25, 26, 27, 28, 29, or 20 nucleotides.

In some embodiments, the hairpin domain is from about 5 to about 20 nucleotides. In some embodiments, the hairpin domain is from about 10 to about 20 nucleotides. In some embodiments, the hairpin domain is from about 15 to about 20 nucleotides. In some embodiments, the hairpin domain is from about 5 to about 15 nucleotides. In some embodiments, the hairpin domain is from about 5 to about 10 nucleotides. In some embodiments, the hairpin domain includes 5, 6, 7, 8, 9, 10, 11, 12, 13, 14, 15, 16, 17, 18, 19, or 20 nucleotides.

In some embodiments, the sensor comprises at least two hydrophobic tags. In some embodiments, the at least two hydrophobic tags are positioned from about 120° to about 180° from each other. In some embodiments, the at least two hydrophobic tags are positioned about 180° from each other. In some embodiments, the at least two hydrophobic tags are positioned from about 4 to about 6 nucleotides from each other. In some embodiments, the at least two hydrophobic tags are positioned from 2, 3, 4, 5, or 6 nucleotides from each other.

In some embodiments, the sensor comprises at least three hydrophobic tags. In some embodiments, the at least three hydrophobic tags are positioned from about 90° to about 120° from each other. In some embodiments, the at least three hydrophobic tags are positioned about 120° from each other. In some embodiments, the at least three hydrophobic tags are positioned from about 2 to about 4 nucleotides from each other. In some embodiments, the at least three hydrophobic tags are positioned from about 1, 2, 3, or 4 nucleotides from each other.

In some embodiments, the at least one hydrophobic tag is positioned such that the at least one hydrophobic tag spans from about 1.0 nm to about 3.0 nm. In some embodiments, the at least one hydrophobic tag is positioned such that the at least one hydrophobic tag spans from about 1.5 nm to about 3.0 nm. In some embodiments, the at least one hydrophobic tag is positioned such that the at least one hydrophobic tag spans from about 2.0 nm to about 3.0 nm. In some embodiments, the at least one hydrophobic tag is positioned such that the at least one hydrophobic tag spans from about 2.5 nm to about 3.0 nm. In some embodiments, the at least one hydrophobic tag is positioned such that the at least one hydrophobic tag spans from about 1.0 nm to about 2.5 nm. In some embodiments, the at least one hydrophobic tag is positioned such that the at least one hydrophobic tag spans from about 1.0 nm to about 2.0 nm. In some embodiments, the at least one hydrophobic tag is positioned such that the at least one hydrophobic tag spans from about 1.0 nm to about 1.5 nm.

In some embodiments, the sensor comprises a nucleic acid sequence that is at least 90% identical to SEQ ID NOs: 1 or 2. In some embodiments, the sensor comprises a nucleic acid sequence that is at least 91% identical to SEQ ID NOs: 1 or 2. In some embodiments, the sensor comprises a nucleic acid sequence that is at least 92% identical to SEQ ID NOs: 1 or 2. In some embodiments, the sensor comprises a nucleic acid sequence that is at least 93% identical to SEQ ID NOs: 1 or 2. In some embodiments, the sensor comprises a nucleic acid sequence that is at least 94% identical to SEQ ID NOs: 1 or 2. In some embodiments, the sensor comprises a nucleic acid sequence that is at least 95% identical to SEQ ID NOs: 1 or 2. In some embodiments, the sensor comprises a nucleic acid sequence that is at least 96% identical to SEQ ID NOs: 1 or 2. In some embodiments, the sensor comprises a nucleic acid sequence that is at least 97% identical to SEQ ID NOs: 1 or 2. In some embodiments, the sensor comprises a nucleic acid sequence that is at least 98% identical to SEQ ID NOs: 1 or 2. In some embodiments, the sensor comprises a nucleic acid sequence that is at least 99% identical to SEQ ID NOs: 1 or 2.

In some embodiments, the sensor comprises a nucleic acid sequence that is at least 90% identical to SEQ ID NOs: 5, 8, or 11. In some embodiments, the sensor comprises a nucleic acid sequence that is at least 91% identical to SEQ ID NOs: 5, 8, or 11. In some embodiments, the sensor comprises a nucleic acid sequence that is at least 92% identical to SEQ ID NOs: 5, 8, or 11. In some embodiments, the sensor comprises a nucleic acid sequence that is at least 93% identical to SEQ ID NOs: 5, 8, or 11. In some embodiments, the sensor comprises a nucleic acid sequence that is at least 94% identical to SEQ ID NOs: 5, 8, or 11. In some embodiments, the sensor comprises a nucleic acid sequence that is at least 95% identical to SEQ ID NOs: 5, 8, or 11. In some embodiments, the sensor comprises a nucleic acid sequence that is at least 96% identical to SEQ ID NOs: 5, 8, or 11. In some embodiments, the sensor comprises a nucleic acid sequence that is at least 97% identical to SEQ ID NOs: 5, 8, or 11. In some embodiments, the sensor comprises a nucleic acid sequence that is at least 98% identical to SEQ ID NOs: 5, 8, or 11. In some embodiments, the sensor comprises a nucleic acid sequence that is at least 99% identical to SEQ ID NOs: 5, 8, or 11.

Embodiments of the present disclosure also include a method of detecting a target nucleic acid using any of the sensors of described herein. In some embodiments, the target nucleic acid is located within a membranous vesicle or cell. In some embodiments, the method comprises detecting the target nucleic acid without lysis of the membranous vesicle or cell. In some embodiments, the target nucleic acid is DNA or RNA.

In some embodiments, detecting the target nucleic acid comprises an amplification step. In some embodiments, the amplification step comprises at least one of toehold mediated strand displacement (TMSD), loop-mediated isothermal amplification (LAMP), and/or hairpin chain reaction (HCR), In some embodiments, the method comprises a target detection step. For example, in some embodiments, nucleic acid detection includes the use of a fluorophore, a chromophore, fluorophore pairs, fluorophore-quencher pairs or other detection moiety known in the art.

In some embodiments, the method comprises sequencing the target nucleic acid. For example, in some embodiments, detection of the target nucleic acid includes sequencing using sanger sequence, next-generation sequencing (NGS), or any other sequencing methods known in the art.

Embodiments of the present disclosure also include a composition comprising any of the nucleic acid sensors described herein. In some embodiments, the composition comprises at least one reporter nucleic acid. In some embodiments, the at least one reporter nucleic acid comprises a sequence that is complementary to at least a portion of the optional hairpin domain. In some embodiments, the at least one reporter nucleic acid comprises a sequence capable of initiating at least one of: (i) toehold mediated strand displacement (TMSD); (ii) loop-mediated isothermal amplification (LAMP); and/or (iii) hairpin chain reaction (HCR).

Embodiments of the present disclosure also include a kit comprising any of the sensors described herein, and instructions for detecting a target nucleic acid. In some embodiments, the kit further comprises a calibrator or control. In some embodiments, the calibrator or control comprises a detection moiety. In some embodiments, the calibrator or control comprises a nucleic acid sequence that is at least 90% identical to SEQ ID NOs: 3, 4, 6, 7, 9, 10, 12, or 13. In some embodiments, the calibrator or control comprises a nucleic acid sequence that is at least 91% identical to SEQ ID NOs: 3, 4, 6, 7, 9, 10, 12, or 13. In some embodiments, the calibrator or control comprises a nucleic acid sequence that is at least 92% identical to SEQ ID NOs: 3, 4, 6, 7, 9, 10, 12, or 13. In some embodiments, the calibrator or control comprises a nucleic acid sequence that is at least 93% identical to SEQ ID NOs: 3, 4, 6, 7, 9, 10, 12, or 13. In some embodiments, the calibrator or control comprises a nucleic acid sequence that is at least 94% identical to SEQ ID NOs: 3, 4, 6, 7, 9, 10, 12, or 13. In some embodiments, the calibrator or control comprises a nucleic acid sequence that is at least 95% identical to SEQ ID NOs: 3, 4, 6, 7, 9, 10, 12, or 13. In some embodiments, the calibrator or control comprises a nucleic acid sequence that is at least 96% identical to SEQ ID NOs: 3, 4, 6, 7, 9, 10, 12, or 13. In some embodiments, the calibrator or control comprises a nucleic acid sequence that is at least 97% identical to SEQ ID NOs: 3, 4, 6, 7, 9, 10, 12, or 13. In some embodiments, the calibrator or control comprises a nucleic acid sequence that is at least 98% identical to SEQ ID NOs: 3, 4, 6, 7, 9, 10, 12, or 13. In some embodiments, the calibrator or control comprises a nucleic acid sequence that is at least 99% identical to SEQ ID NOs: 3, 4, 6, 7, 9, 10, 12, or 13.

### 3. Sequences

The various nucleic acid sequences referenced (i.e., SEQ ID NOs) herein are provided below.

TABLE 1

| Double-Stranded DNA | |
|---|---|
| Sensor Strands | Sequence |
| Strand 1 | GTTTTTGTTTGTAGGAGT/iCholTEG/GAGTT/iCholTEG/ |
| | GAATG (SEQ ID NO: 1) |
| Strand 2 | GTTTTTGTTTGTACATTCAA/iCholTEG/CTCAC/iCholTEG/ |
| | TCC (SEQ ID NO: 2) |
| Target Strand | CCCTACTCACCTCCACATTCAACTCACTCCTACAAA |
| | CAAAAAC/3Cy5/ (SEQ ID NO: 3) |
| Signal/Reporter Strand | TGGAGGTGAGTAGGG/3Atto488/ (SEQ ID NO: 4) |

TABLE 2

| Hairpin Single-Stranded | |
|---|---|
| DNA Sensors | Sequence |
| Sensor strand with synthetic | GTTTTTGTTTGTAGGA/iCholTEG/GTG/iCholTEG/ |
| target (hairpin sensor a) | AGTT/iCholTEG/ |
| | GAATGAGATTACGTTTAGCATTCAACTCACTCC |
| | (SEQ ID NO: 5) |
| Synthetic Target a (target for | CATTCAACTCACTCCTACAAACAAAAAC/3Cy3/ |
| hairpin sensor a) | (SEQ ID NO: 6) |
| Signal/Reporter strand hairpin | /5Cy5/GGAGTGAGTTGAATGCTAAACGTGAACGGAC |
| sensor a | GTTTAGCATTCAA (SEQ ID NO: 7) |
| Sensor strand with miR23b | TGC CAG GGA TTA CCT AAC CTC GGT |
| target (hairpin sensor b) | A/iCholTEG/A TCC/iCholTEG/CT G/iCholTEG/G CAA |
| | TGT GAT (SEQ ID NO: 8) |
| miR23b target | /5Atto488/ATCACATTGCCAGGGATTACC |
| | (SEQ ID NO: 9) |
| Signal strand hairpin sensor b | GGTAATCCCTGGCA/3Cy5/ (SEQ ID NO: 10) |
| Sensor strand with synthetic | CGGAGGTTGAGAGTGGATGGAGT/iCholTEG/GAGTT/ |
| target (hairpin sensor c with 31 | iCholTEG/GAATGAGTGGATGAATCTAACCGCATCC |
| stem) | ACTCATTCAACTCACTCCATCCACTC |
| | (SEQ ID NO: 11) |
| Synthetic Target c (target for | CATCCACTCATTCAACTCACTCCATCCACTCTCAAC |
| hairpin sensor c) | CTCCG\Cy3\ (SEQ ID NO: 12) |
| Signal/reporter strand hairpin | GAGTGAGTTGAAT\Cy5\ (SEQ ID NO: 13) |
| sensor c | |

It will be readily apparent to those skilled in the art that other suitable modifications

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

### 4. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Sensor design, structure and working principle. In one embodiment, the single stranded DNA sensor is composed of toehold, stem, and hairpin sections. The duplex DNA has two toeholds at each end, connected by a stem section. One or two strands are required for this embodiment, which is very powerful at overcoming stoichiometric problems during the formation of sensors, which is often a challenge for DNA nanotechnology. Additionally, this simple approach will benefit the insertion kinetics and number of hydrophobic tags that are needed.

Based on the target length, the toehold can be about 6-13 nucleotides that are complementary to the part of the internal nucleic acid target to be detected. The stem is designed to provide structural integrity to the hairpin and duplex structure. The toehold mediates the displacement of the stem section, allowing the signal to be transduced from the inside to the outside. This stem has about 14-15 base pairs, depending on the target length, which are either the same or complementary to the target sequence. The hairpin in single stranded sensors is designed to trigger an amplification of isothermal amplification, such as HCR or other methods such as rolling circle amplification. In order to maintain polar group balance on both sides of the stem, the hairpin is chosen to be the same length as the toehold. Hydrophobic modifications are strategically placed on one side of the stem. Consequently, the other side of the stem can be detached from the membrane once the TMSD is complete. FIGS. 1A-1B shows the schematic of the DNA sensor designs.

In order for the DNA sensor to cover hydrophobic liquids efficiently, the spatial arrangement of the hydrophobic tags (either two or three tags) is carefully designed in such a way that when projected onto the cross section of the stem, the distance between them is 180 degrees for two hydrophobic tags or 120 degrees for three hydrophobic tags (see FIGS. 1A and 1B, middle panel). Accordingly, the angular separation between two tags is achieved by 5 nucleotides in the case of two tags and 3 and 4 nucleotides in the case of three tags. This can be seen in FIG. 1A and FIG. 1B, left panels, where the hydrophobic tags are shown in green and the spacing between them also shown. As a result, the stem will be anchored perpendicular to the membrane surface inside the lipid bilayer membrane. Based on the separation of 5 nucleotides and 3-4 nucleotides, the distance covered by the hydrophobic tags is around 1.8 to 2.5 nm, which is less than the known thickness of lipid bilayers of 3.5-4.0 nm, which ensures the stem sits perpendicularly in the membrane, and the hairpin and toehold are free on opposite sides.

The hydrophobic tags are asymmetrically placed along the stem to favor insertion of the toehold into the vesicle for successful internal nucleic acid detection. As a result, the hydrophobic group is positioned closer to the toehold region so that it gives a less polar nucleic acid on the toehold stem side. Given that the toehold and hairpin are the same length, the orientation during insertion only depends on the position of the hydrophobic group on the stem, which is very versatile in terms of design. The placement of the hydrophobic tags is shown in FIGS. 1A and 1B, left panels.

Two mismatches are introduced on the 14-15 base pair-long stem to create faster kinetics of TMSD, along with an optimal toehold length of around 6-10 nt (Zhang and Winfree, 2009). The mismatches are carefully chosen and placed so they will not affect the integrity of the stem structure, but only help the kinetics of TMSD. The mismatches are located in the vicinity of the hydrophobic group. Nupack shows a high probability of equilibrium around the mismatches, thus confirming the stem's integrity, but decreasing the region's stability.

Once the TMSD is completed, the signal is transduced across the membrane. As a result of being displaced by the target strand, one of the single strand units of the stem region will be freed from membrane confinement to be outside the vesicle. As the opened single stranded hairpin section is displaced by the single stranded unit of the stem, a hairpin chain reaction is triggered. FIG. 1B right panel shows two monomer hairpins of HCR in the case of single stranded DNA sensor. They are designed specifically in such a way that the reaction will only be triggered if there is a successful event of internal target detection and TMSD across the lipid bilayer membrane. For double-stranded sensors, the internal target strand is designed to have a section that acts as a target for the signal strands with fluorophores to bind and detect the TMSD. HCR cannot be carried out for the double stranded DNA sensor due to the length limitation of the target, FIG. 1A shows the internal target and the signal strand for the double stranded DNA sensor.

In the case of double stranded DNA, the signal strand will bind to part c shown in FIG. 1A. For hairpin sensor, HCR amplification will occur once the hairpin is opened. The binding of the signal strand or the monomers of HCR allows detection of the fluorophore signal, which may be detected by microscopy or gel assay. In microscopy, the signal strand and one of the hairpin monomers can be conjugated with a fluorophore to detect the polymer formed from HCR. It is also possible to monitor the detection at the plate reader using monomer hairpins with an emitter-quencher pair or a donor-acceptor pair, or by using fluorescent dequenching detection or FRET.

Using the design principles above, Nupack is then used to design and generate optimized sequences for single and double stranded DNA sensors. Among the initial targets is the sequence from microRNA miR23b, an adaptation for practical application. DNA sensors are listed in Tables 1 and 2, along with their targets, signal strands for double stranded sensors and hairpin monomers for single stranded sensors.

Giant unilamellar vesicles (GUVs) with phospholipids and some cholesterol in their membranes were used to carry out proof-of-concept experiments. GUVs are synthesized with target DNA encapsulated within them. Using fluorescence confocal microscopy, the DNA target and the DNA sensor will both be labeled with fluorescence dye to enable the analysis of the DNA target's interaction with the GUV

### Example 2

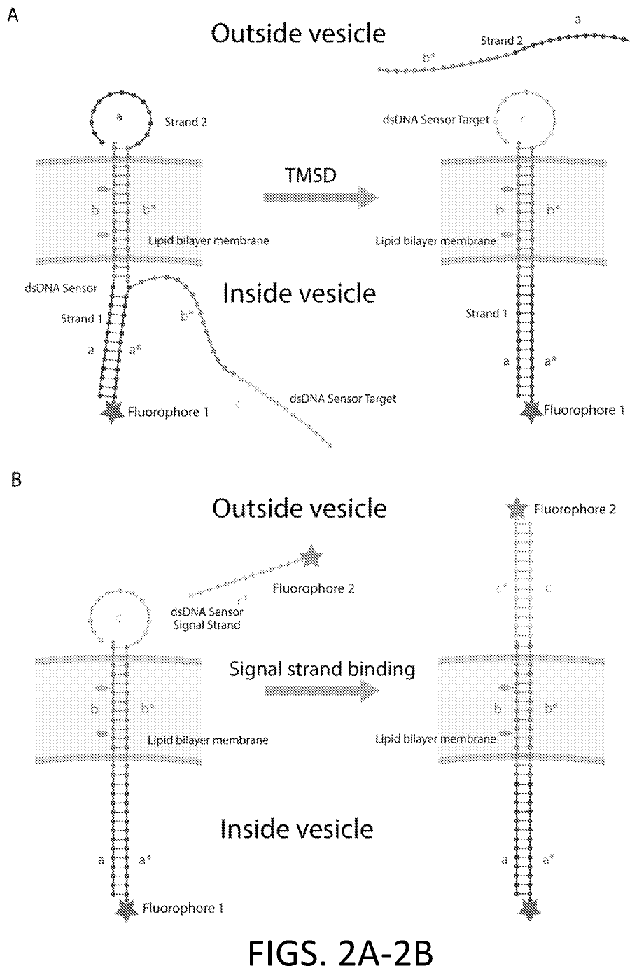

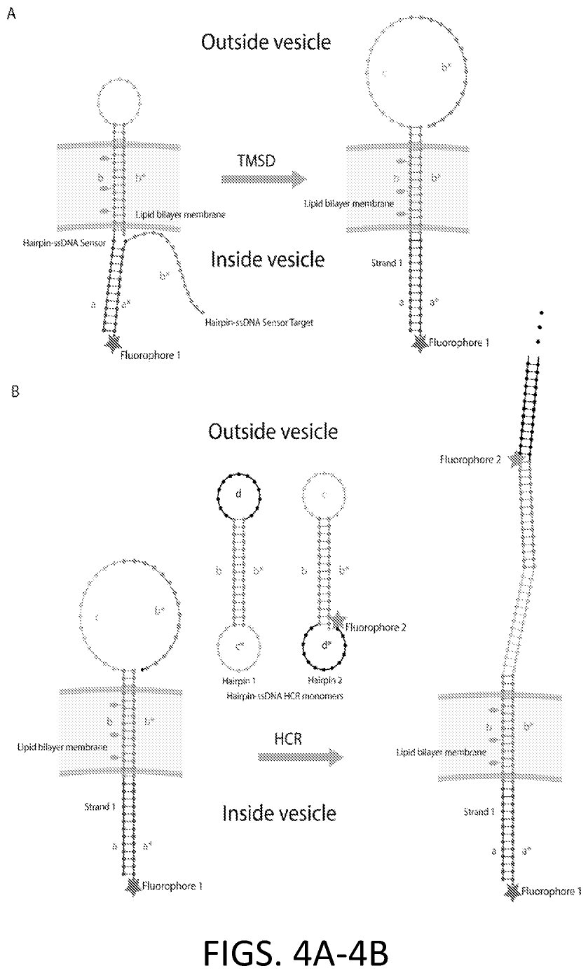

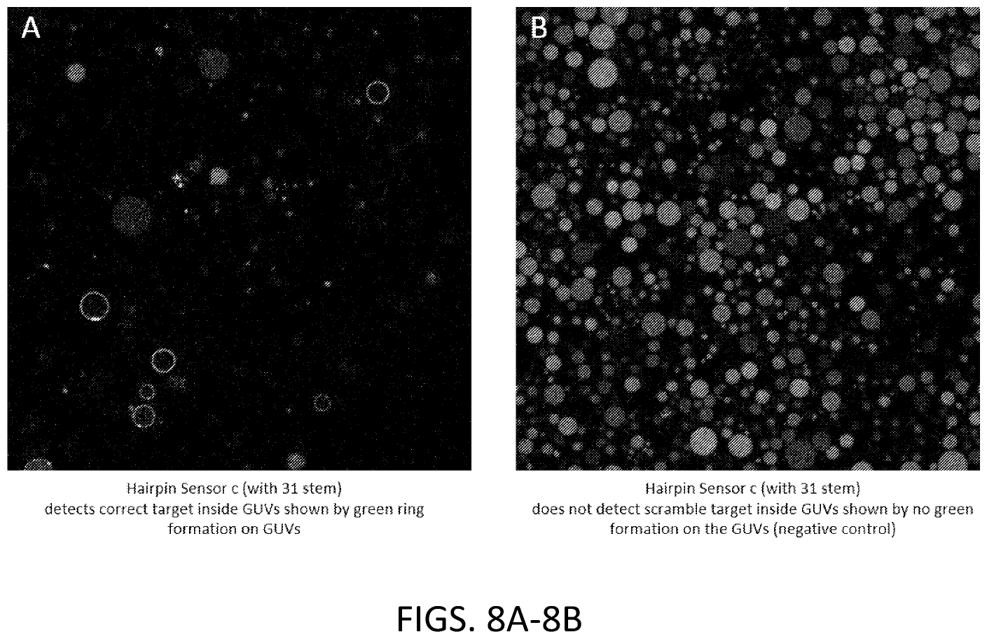

Double-stranded and hairpin DNA sensors insertion across a membrane and detection of single stranded DNA within a GUV as a proof of concept for DNA sensor insertion across the membrane. Experiments were conducted to demonstrate that the DNA sensors can insert the lipid bilayer membrane and sense the internal target inside the vesicles. In order to demonstrate how the insertion and detection work, a very simple vesicle was synthesized from minimal lipid composition of POPC and cholesterol. The left panels of FIGS. 2A and 4A show a schematic of the process once the target binds to sensors containing double-stranded and single-stranded DNA. FIG. 8A shows successful intravesicular target detection by hairpin DNA sensor forming green ring on the GUVs and FIG. 8B is the negative control by having scramble intravesicular DNA target where no green ring is observed.

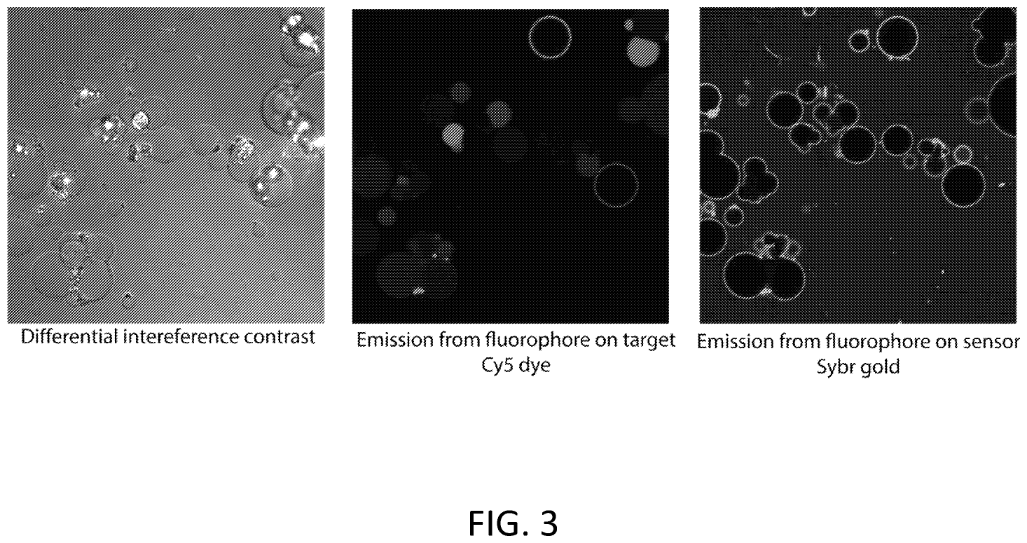

By using a test sample of GUV encapsulating a specific target DNA that is complementary to the toehold of the single and double stranded DNA sensors, the detection of internal nucleic acid targets is demonstrated. This GUV is synthesized by reverse emulsion or cDICE method, where POPC phospholipid and cholesterol have a ratio of 70%:30%, respectively. The inner solution contains sucrose of a certain concentration, as well as KCl of 250 mM and a target concentration of 100-1000 nm. There is an outer solution of glucose of a certain concentration and KCl of 250 mM. The concentration of sucrose and glucose is adjusted to equalize the osmolarity of the inner and outer solutions. The DNA sensors are then mixed with the test sample and incubated for about 1.5 hours before being examined under a confocal microscope. In FIGS. 1A-1B, the DNA sensors are labelled with sybr gold, while the internal target DNA is tagged with Cy5 fluorophore, referred to as fluorophore 1. FIG. 3 shows confocal images showing the insertion of double stranded DNA sensors through the membrane showing green rings on the GUVs on the right panel and the detection of target strands forming red rings on the GUVs on the middle panel. The insertion and detection are checked using a fluorescent confocal microscope experiment.

### Example 3

Toehold mediated strand displacement across GUV membranes. Toehold mediated strand displacement (TMSD) occurs in solution when there is no confining membrane surrounding the DNA (Yurke et al, 2000). In this embodiment, the DNA sensors undergo TMSD under lipid bilayer membrane confinement. In the design section, it was noted that the sensors have hydrophobic tags on one side of the double stranded stem, allowing the other side to be released to the outside of the vesicle once TMSD is completed across the membrane. TMSD across the membrane will have zero differences in total Gibbs free energy from the perspective of a single base pair. TMS's kinetics is expected to be slower than that in solution only if there is no confining membrane. FIGS. 2A and 4A right panel show the schematic for the single stranded and double stranded DNA sensors.

The GUV test sample described in section II is used. It is tagged with a signal strand that has a different fluorophore from the target. In the case of single stranded DNA sensors, the signal strand is designed with Nupack (nupack.org) to ensure no leakage will occur if TMSD across the membrane does not happen. Additionally, the assays shown in FIGS. 2A and 4A are carefully designed so that no false positives can be triggered. Upon completion of the TMSD across membrane, the detection of internal target information is transduced outside and the signal strand is expected to bind with the part of the sensor that is released to the outside yet still anchored to the membrane through the sensors in order to give out a fluorescent signal from the sensor surface, as shown in FIG. 2B for double stranded DNA sensors. The event was investigated using fluorescent confocal microscopy.

### Example 4

HCR amplification of internal target detection in hairpin sensor. HCR amplification is used to demonstrate the amplification of detection events. Nupack (nupack.org) was used to design the hairpin monomers. The hairpins and assay are checked for leakage and false positive signals. The schematic for the experiment is shown in FIG. 4B. One of the hairpins is labeled with a different fluorophore than the target. As a result of HCR amplification, the sensor should be able to detect low concentrations of DNA targets. The same GUV test sample is mixed with the sensor and both monomer hairpins. The amplification is observed using fluorescent confocal microscopy.

### Example 5

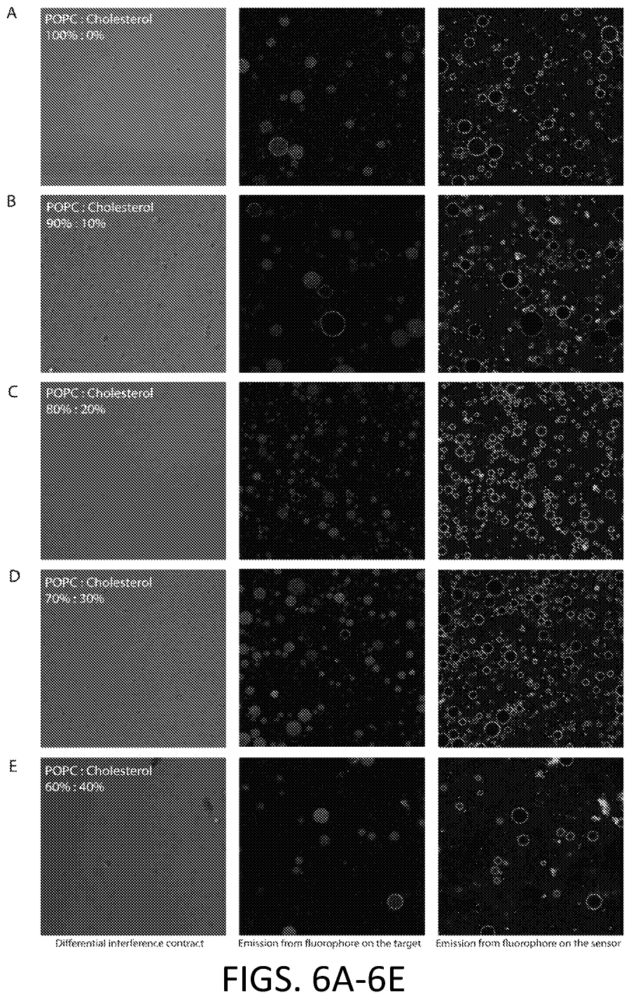

Testing on several GUV composition ratio for double stranded sensor. Experiments were conducted to check the double stranded sensor ability to insert and detect internal nucleic acid targets in the vesicle with several composition ratios of POPC and Cholesterol. These experiments are to test the ability of the double stranded sensor to detect targets encapsulated in different lipid-bilayer membrane composition. FIG. 6 shows red rings as a result of internal nucleic acid detection which proves the double stranded sensor is still functioning very well under different synthetic lipid bilayer composition.

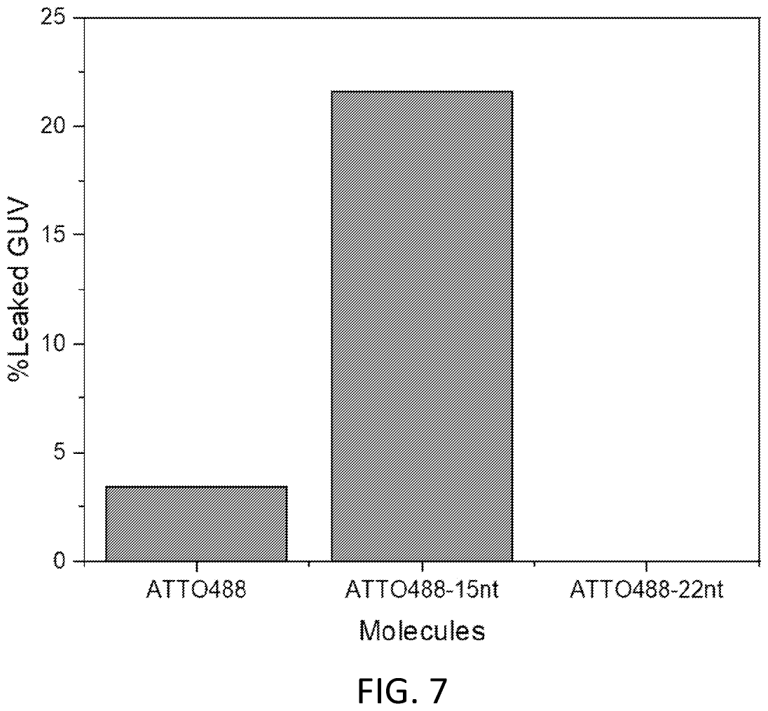

Small dye molecules leakage assay and invasiveness characterization for double stranded sensor. The invasiveness of the double stranded sensor is also checked by performing small dye molecules leakage assay. Confocal images indicated that the sensor is not causing any visible damage to the GUV. However, a leakage assay was performed to test how it affects very small dye molecules only and conjugated with a certain length of single strand DNAs inside GUV which are unrelated to the sensor. The leakage of ATTO488 dye molecules, ATTO488-15nt single strand DNA, and ATTO488-22nt single strand DNA were tested. FIG. 7 depicts the effect it has on the small dye molecules. From the result, it was concluded that the double stranded sensors have minimal effects on unrelated molecules present in GUV, causing only on average around 8.4% leakage in all three experiments.

### Example 6

TMSD across lipid bilayer membrane on double stranded and hairpin DNA sensors possibility. Experiments were conducted to check the possibility of TMSD across lipid membrane on double stranded and hairpin DNA sensors. Inside GUVs, corresponding DNA targets for double stranded and hairpin DNA sensors are encapsulated. The sensors are then added and incubated for several hours to allow TMSD to proceed. Finally, the reporter specific to sensors are added to probe the occurrence of TMSD.

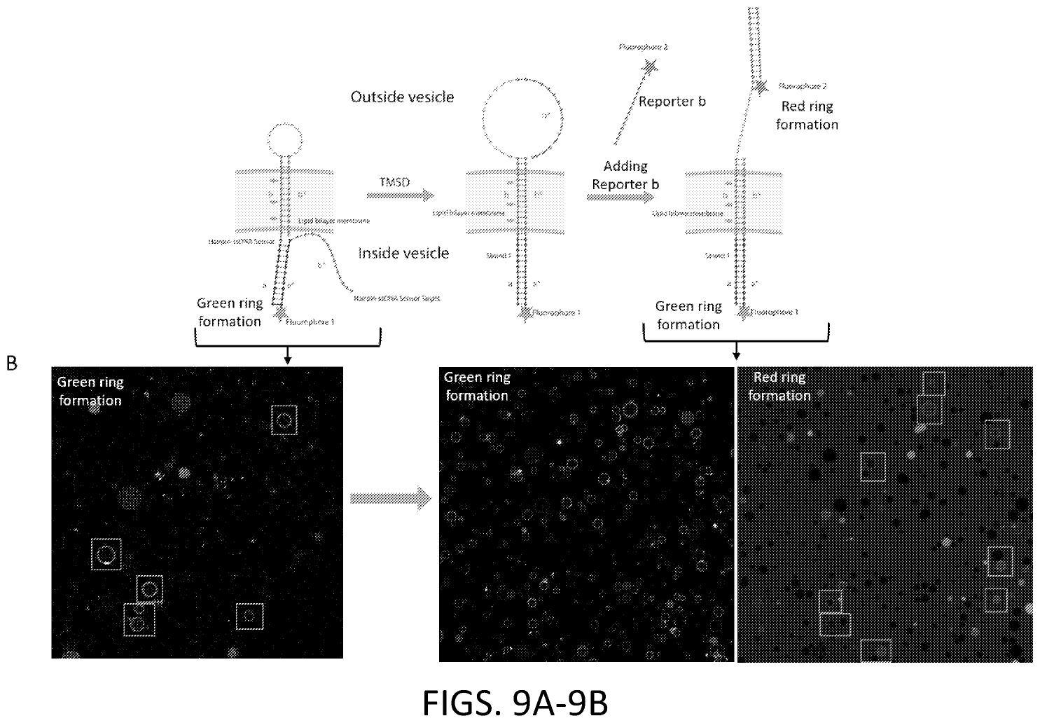

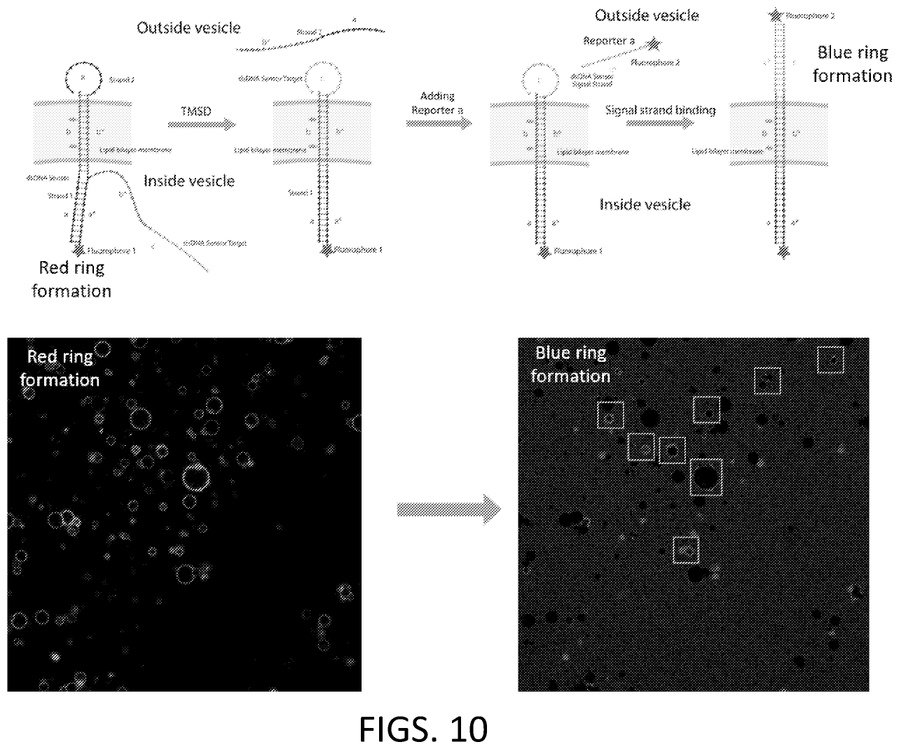

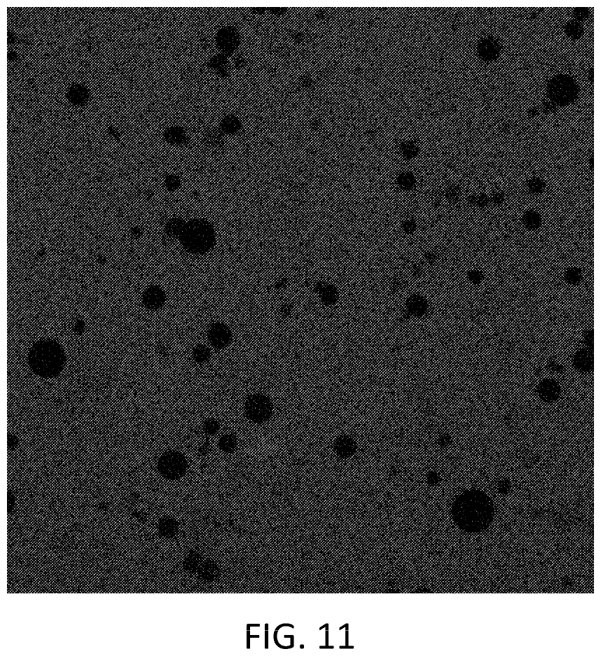

FIG. 9A shows the schematic of the experiment and process for hairpin DNA sensor. FIG. 9B shows the detection of the intravesicular target forming green rings on GUVs on the left panel and the binding of the reporter strands from outside GUVs onto the open stem part of the sensor forming red rings on GUVs. In the case of double stranded DNA sensors, FIG. 10 top panel shows the schematic of the process of the TMSD across membrane and the penetration of some part of the target rendering it available for reporter to bind from outside GUV and bottom panel shows the representative of confocal images where the target detection by double stranded sensors forms red ring on GUVs on left bottom panel and the formation of blue ring on GUVs due to the binding of reporter strands on the sensor on bottom right panel. The negative control experiment by having scramble intravesicular DNA target and using double stranded DNA sensors shows no formation of blue ring on the GUVs as shown in FIG. 11.

It is understood that the foregoing detailed description and accompanying examples are merely illustrative and are not to be taken as limitations upon the scope of the embodiments of the present disclosure, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the embodiments of the present disclosure, may be made without departing from the spirit and scope thereof.

For reasons of completeness, various aspects of the embodiments of the present disclosure are set out in the following numbered clauses:

Clause 1. A nucleic acid sensor comprising: a double-stranded stem domain comprising at least one hydrophobic tag; at least one toehold domain positioned at an end of the double-stranded stem domain; and optionally, a hairpin domain positioned at an end of the double-stranded stem domain opposite of the toehold domain.

Clause 2. The sensor of clause 1, wherein the sensor comprises two toehold domains positioned at both ends of the double-stranded stem domain.

Clause 3. The sensor of clause 1 or clause 2, wherein the sensor does not comprise a hairpin domain.

Clause 4. The sensor of any of clauses 1 to 3, wherein the sensor comprises two separate nucleic acid molecules having complementary sequences that form the double-stranded stem domain, and wherein each of the separate nucleic acid molecules comprises a toehold domain.

Clause 5. The sensor of clause 1, wherein the sensor comprises one toehold domain and one hairpin domain at opposite ends of the double-stranded stem domain.

Clause 6. The sensor of clause 5, wherein the sensor comprises a single nucleic acid molecule, and wherein the single nucleic acid molecule comprises an internally complementary sequence that forms the double-stranded stem domain.

Clause 7. The sensor of any of clauses 1 to 6, wherein the nucleic acid sensor is a DNA molecule, an LNA molecule, or combinations thereof.

Clause 8. The sensor of any of clauses 1 to 6, wherein the nucleic acid sensor is an RNA molecule.

Clause 9. The sensor of any of clauses 1 to 8, wherein the at least one toehold domain is complementary to a target nucleic acid sequence.

Clause 10. The sensor of clause 10, wherein the target nucleic acid sequence is a DNA molecule or an RNA molecule.

Clause 11. The sensor of any of clauses 1 to 10, wherein the at least one toehold domain is from about 5 to about 20 nucleotides.

Clause 12. The sensor of any of clauses 1 to 11, wherein the stem domain is from about 5 to about 30 nucleotides.

Clause 13. The sensor of any of clauses 1 to 12, wherein the hairpin domain is from about 5 to about 20 nucleotides.

Clause 14. The sensor of any of clauses 1 to 13, wherein the sensor comprises at least two hydrophobic tags.

Clause 15. The sensor of clause 14, wherein the at least two hydrophobic tags are positioned from about 120° to about 180° from each other.

Clause 16. The sensor of clause 14 or clause 15, wherein the at least two hydrophobic tags are positioned from about 4 to about 6 nucleotides from each other.

Clause 17. The sensor of any of clauses 1 to 13, wherein the sensor comprises at least three hydrophobic tags.

Clause 18. The sensor of clause 17, wherein the at least three hydrophobic tags are positioned from about 90° to about 120° from each other.

Clause 19. The sensor of clause 17 or clause 18, wherein the at least three hydrophobic tags are positioned from about 2 to about 4 nucleotides from each other.

Clause 20. The sensor of any of clauses 1 to 19, wherein the at least one hydrophobic tag is positioned such that the at least one hydrophobic tag spans from about 1.0 nm to about 3.0 nm.

Clause 21. The sensor of any of clauses 1 to 20, wherein the sensor comprises a nucleic acid sequence that is at least 90% identical to SEQ ID NOs: 1 or 2.

Clause 22. The sensor of any of clauses 1 to 20, wherein the sensor comprises a nucleic acid sequence that is at least 90% identical to SEQ ID NOs: 5, 8, or 11.

Clause 23. A composition comprising the nucleic acid sensor of any one of clauses 1 to 22.

Clause 24. The composition of clause 23, wherein the composition further comprises at least one reporter nucleic acid, wherein the at least one reporter nucleic acid comprises a sequence that is complementary to at least a portion of the optional hairpin domain.

Clause 25. The composition of clause 23, wherein the composition further comprises at least one reporter nucleic acid, wherein the at least one reporter nucleic acid comprises a sequence capable of initiating at least one of: (i) toehold mediated strand displacement (TMSD); (ii) loop-mediated isothermal amplification (LAMP); and/or (iii) hairpin chain reaction (HCR).

Clause 26. A method of detecting a target nucleic acid using any of the sensors of clauses 1 to 22, or the compositions of clauses 23 to 25.

Clause 27. The method of clause 26, wherein the target nucleic acid is located within a membranous vesicle or cell.

Clause 28. The method of clause 27, wherein the method comprises detecting the target nucleic acid without lysis of the membranous vesicle or cell.

Clause 29. The method of any of clauses 26 to 28, wherein the target nucleic acid is DNA or RNA.

Clause 30. The method of any of clauses 26 to 28, wherein detecting the target nucleic acid comprises an amplification step.

Clause 31. The method of clause 30, wherein the amplification step comprises at least one of toehold mediated strand displacement (TMSD), loop-mediated isothermal amplification (LAMP), and/or hairpin chain reaction (HCR).

Clause 32. The method of any of clauses 26 to 31, wherein the method comprises a target detection step.

Clause 33. The method of any of clauses 26 to 32, wherein the method comprises sequencing the target nucleic acid.

Clause 34. A kit comprising any of the sensors of clauses 1 to 22, and instructions for detecting a target nucleic acid.

Clause 35. The kit of clause 34, further comprising a calibrator or control.

Clause 36. The kit of clause 35, wherein the calibrator or control comprises a detection moiety.

Clause 37. The kit of clause 35, wherein the calibrator or control comprises a nucleic acid sequence that is at least 90% identical to SEQ ID NOs: 3, 4, 6, 7, 9, 10, 12, or 13.

What is claimed is:

1. A nucleic acid sensor comprising:
a double-stranded stem domain comprising at least one hydrophobic tag;
at least one toehold domain positioned at an end of the double-stranded stem domain; and
optionally, a hairpin domain positioned at an end of the double-stranded stem domain opposite of the toehold domain.

2. The sensor of claim 1, wherein the sensor comprises two toehold domains positioned at both ends of the double-stranded stem domain.

3. The sensor of claim 1 or claim 2, wherein the sensor does not comprise a hairpin domain.

4. The sensor of claim 1, wherein the sensor comprises two separate nucleic acid molecules having complementary sequences that form the double-stranded stem domain, and wherein each of the separate nucleic acid molecules comprises a toehold domain.

5. The sensor of claim 1, wherein the sensor comprises one toehold domain and one hairpin domain at opposite ends of the double-stranded stem domain.

6. The sensor of claim 5, wherein the sensor comprises a single nucleic acid molecule, and wherein the single nucleic acid molecule comprises an internally complementary sequence that forms the double-stranded stem domain.

7. The sensor of claim 1, wherein the nucleic acid sensor is a DNA molecule, an LNA molecule, or combinations thereof.

8. The sensor of claim 1, wherein the nucleic acid sensor is an RNA molecule.

9. The sensor of claim 1, wherein the at least one toehold domain is complementary to a target nucleic acid sequence.

10. The sensor of claim 10, wherein the target nucleic acid sequence is a DNA molecule or an RNA molecule.

11. The sensor of claim 1, wherein the at least one toehold domain is from about 5 to about 20 nucleotides.

12. The sensor of claim 1, wherein the stem domain is from about 5 to about 30 nucleotides.

13. The sensor of claim 1, wherein the hairpin domain is from about 5 to about 20 nucleotides.

14. The sensor of claim 1, wherein the sensor comprises at least two hydrophobic tags.

15. The sensor of claim 14, wherein the at least two hydrophobic tags are positioned from about 120° to about 180° from each other.

16. The sensor of claim 14, wherein the at least two hydrophobic tags are positioned from about 4 to about 6 nucleotides from each other.

17. The sensor of claim 1, wherein the sensor comprises at least three hydrophobic tags.

18. The sensor of claim 17, wherein the at least three hydrophobic tags are positioned from about 90° to about 120° from each other.

19. The sensor of claim 17, wherein the at least three hydrophobic tags are positioned from about 2 to about 4 nucleotides from each other.

20. The sensor of claim 1, wherein the at least one hydrophobic tag is positioned such that the at least one hydrophobic tag spans from about 1.0 nm to about 3.0 nm.

21. The sensor of claim 1, wherein the sensor comprises a nucleic acid sequence that is at least 90% identical to SEQ ID NOs: 1 or 2.

22. The sensor of claim 1, wherein the sensor comprises a nucleic acid sequence that is at least 90% identical to SEQ ID NOs: 5, 8, or 11.

23. A composition comprising the nucleic acid sensor of claim 1.

24. The composition of claim 23, wherein the composition further comprises at least one reporter nucleic acid, wherein the at least one reporter nucleic acid comprises a sequence that is complementary to at least a portion of the optional hairpin domain.

25. The composition of claim 23, wherein the composition further comprises at least one reporter nucleic acid, wherein the at least one reporter nucleic acid comprises a sequence capable of initiating at least one of: (i) toehold mediated strand displacement (TMSD); (ii) loop-mediated isothermal amplification (LAMP); and/or (iii) hairpin chain reaction (HCR).

26. A method of detecting a target nucleic acid using any of the sensors of claim 1, or the composition of claim 23.

27. The method of claim 26, wherein the target nucleic acid is located within a membranous vesicle or cell.

28. The method of claim 27, wherein the method comprises detecting the target nucleic acid without lysis of the membranous vesicle or cell.

29. The method of claim 26, wherein the target nucleic acid is DNA or RNA.

30. The method of claim 26, wherein detecting the target nucleic acid comprises an amplification step.

31. The method of claim 30, wherein the amplification step comprises at least one of toehold mediated strand displacement (TMSD), loop-mediated isothermal amplification (LAMP), and/or hairpin chain reaction (HCR).

32. The method of claim 26, wherein the method comprises a target detection step.

33. The method of claim 26, wherein the method comprises sequencing the target nucleic acid.

34. A kit comprising any of the sensors of claim 1, and instructions for detecting a target nucleic acid.

35. The kit of claim 34, further comprising a calibrator or control.

36. The kit of claim 35, wherein the calibrator or control comprises a detection moiety.

37. The kit of claim 35, wherein the calibrator or control comprises a nucleic acid sequence that is at least 90% identical to SEQ ID NOs: 3, 4, 6, 7, 9, 10, 12, or 13.


SEQUENCE LISTING (13 sequences)

SEQ ID NO: 1 - DNA, 28 bp, Synthetic construct
  source 1..28: mol_type = other DNA
gtttttgtttgtaggagtgagttgaatg

SEQ ID NO: 2 - DNA, 28 bp, Synthetic construct
  source 1..28: mol_type = other DNA
gtttttgtttgtacattcaactcactcc

SEQ ID NO: 3 - DNA, 43 bp, Synthetic construct
  source 1..43: mol_type = other DNA
ccctactcacctccacattcaactcactcctacaaacaaaaac

SEQ ID NO: 4 - DNA, 15 bp, Synthetic construct
  source 1..15: mol_type = other DNA
tggaggtgagtaggg

SEQ ID NO: 5 - DNA, 56 bp, Synthetic construct
  source 1..56: mol_type = other DNA
gtttttgtttgtaggagtgagttgaatgagattacgtttagcattcaactcactcc

SEQ ID NO: 6 - DNA, 28 bp, Synthetic construct
  source 1..28: mol_type = other DNA
cattcaactcactcctacaaacaaaaac

SEQ ID NO: 7 - DNA, 44 bp, Synthetic construct
  source 1..44: mol_type = other DNA
ggagtgagttgaatgctaaacgtgaacggacgtttagcattcaa

SEQ ID NO: 8 - DNA, 42 bp, Synthetic construct
  source 1..42: mol_type = other DNA
tgccagggattacctaacctcggtaatccctggcaatgtgat

SEQ ID NO: 9 - DNA, 21 bp, Synthetic construct
  source 1..21: mol_type = other DNA
atcacattgccagggattacc

SEQ ID NO: 10 - DNA, 14 bp, Synthetic construct
  source 1..14: mol_type = other DNA
ggtaatccctggca

SEQ ID NO: 11 - DNA, 82 bp, Synthetic construct
  source 1..82: mol_type = other DNA
cggaggttgagagtggatggagtgagttgaatgagtggatgaatctaaccgcatccactcattcaactcactccatccactc

SEQ ID NO: 12 - DNA, 41 bp, Synthetic construct
  source 1..41: mol_type = other DNA
catccactcattcaactcactccatccactctcaacctccg

SEQ ID NO: 13 - DNA, 13 bp, Synthetic construct
  source 1..13: mol_type = other DNA
gagtgagttgaat